(19) United States
(12) Patent Application Publication
(10) Pub. No.: US 2005/0238622 A1
(43) Pub. Date: Oct. 27, 2005

(54) Compositions and methods for treating cancer with an oncolytic viral agent

(76) Inventors: Jonathan H. Axelrod, Jerusalem (IL); Eithan Galun, Har Adar (IL); Stefan Rose-John, Schellhorn (DE)

Correspondence Address: WINSTON & STRAWN LLP, 1700 K STREET, N.W., WASHINGTON, DC, 20006, US

(21) Appl. No.: 10/978,278

(22) Filed: Oct. 28, 2004

Related U.S. Application Data
(63) Continuation of application No. PCT/IL03/00343, filed on Apr. 29, 2003.
(60) Provisional application No. 60/375,801, filed on Apr. 29, 2002.

(51) Int. Cl. A61K 48/00

(52) U.S. Cl. 424/93.2; 514/44

(57) ABSTRACT

The invention discloses lytic viruses as anti-neoplastic agents for specifically replicating and lysing tumor cells. According to the present invention, the agents preferably include E1A deficient adenoviral vectors, exemplified by Ad.HIL6gfp, encoding an IL-6/sIL-6R complex, HIL-6, which is able to replicate, produce cytotoxic effects, and kill tumor cells in the absence of either E1A or exogenous IL-6 protein. These viral agents have utility as therapeutic vehicles for treating cancers of various types either as a single agent, or applied in combination with other therapeutic strategies.

### CROSS-REFERENCE TO RELATED APPLICATIONS

This application is a continuation of International application PCT/IL03/00343 filed Apr. 29, 2003 and claims the benefit of U.S. provisional application 60/375,801 filed Apr. 29, 2002, the entire content of each of which is expressly incorporated herein by reference thereto.

### FIELD OF THE INVENTION

The present invention discloses oncolytic viral agents and in particular, the use of recombinant adenoviral vectors encoding an IL-6/sIL-6R complex, the expression of which is regulated by an appropriate promoter, and which is able to replicate and lyse tumor cells.

### BACKGROUND OF THE INVENTION

The use of lytic viruses as anti-neoplastic agents for experimental tumor therapy and also in the clinical setting has been studied for more than 50 years. Because of their apparent oncolytic properties, mumps virus, vaccinia virus, myxovirus, West Nile Virus, and Newcastle Disease Virus, were used in early studies for the treatment of different neoplastic diseases. More recent studies with H-1 parvovirus, Newcastle Disease Virus, Measles Virus, and Herpes Simplex Virus type 1 have demonstrated that tumor cell killing and regression of tumors can be achieved by viral-mediated oncolysis. Wild-type adenoviruses, which also possess a lytic life cycle, were explored as oncolytic agents soon after their discovery in the 1950's, with only local effects observed.

It was demonstrated that replication deficient recombinant adenoviruses could be produced by replacing the E1 region necessary for replication with a target gene. More recently it was shown that mutations in the adenovirus E1B gene caused the virus to replicate selectively in p53-defective tumor cells (1). This observation led to the development of an anti-tumor product, Onyx-015, which has since been developed for commercial use and tested in a number of clinical trials. Other strategies for the selective elimination of tumor cells have been developed based on first generation E1/E3 deleted, replication defective, recombinant adenoviral vectors that have been designed to deliver therapeutic genes (2). In such strategies, vectors have been engineered to deliver various functional DNAs including: (i) genes which sensitize the tumor cells to anti-tumour agents, such as non-toxic pro-drugs (e.g., HSV-tk), or ionizing irradiation (e.g., p53); (ii) tumor suppressor genes (e.g., p53 or Rb) or (iii) genes thought to enhance an innate immune response to the tumor cells (e.g., interleukins, GM-CSF, etc.). A wide variety of strategies have been developed, and many have succeeded to enter clinical trials, some of which having progressed to advanced phases (2).

Recombinant adenoviruses have distinct advantages over retroviral and other gene delivery methods. Adenoviruses have never been shown to induce tumors in humans and have been safely used as live vaccines. Adenovirus does not integrate into the human genome as a normal consequence of infection, thereby greatly reducing the risk of insertional mutagenesis possible with retrovirus or adeno-associated viral (AAV) vectors. This lack of stable integration also leads to an additional safety feature in that the transferred gene effect will be transient, as the extrachromosomal DNA will be gradually lost with continued division of normal cells. Stable, high titer recombinant adenovirus can be produced at levels not achievable with retrovirus or AAV, allowing enough material to be produced to treat a large patient population. Moreover, adenovirus vectors have been shown to be capable of highly efficient in vivo gene transfer into a broad range of tissue and tumor cell types.

In 1991, Spergel and Chen-Kiang reported the observation of an interleukin-6 (IL-6) inducible cellular function in HepG2 cells that functionally substitutes for the adenoviral E1A proteins (3). This activity effectively allows E1A deficient viruses, such as Ad5d1312, to replicate to wild-type levels in certain cell types. This IL-6-inducible E1A-substituting activity was subsequently identified as NF-IL-6, a member of the C/EBP family (4).

Rancourt, et al., subsequently showed that Ad5d1312 could replicate in tumor cells possessing an IL-6 autocrine arc, such as ovarian tumor cells, and kill tumor cells in the absence of exogenous IL-6 (5). In contrast, normal human mesothelial cells did not support viral replication, even in the presence of exogenous IL-6, suggesting that such viruses may be used as specific cytotoxic agents to selectively kill tumor cells responsive to or possessing an IL-6 autocrine arc (5).

IL-6 is a member of a family of cytokines that act via receptor complexes containing at least one subunit of the transmembrane signal transducing protein, gp130, which is found in almost all organs, including heart, kidney, spleen, liver, lung and brain. On target cells, IL-6 acts by binding to a specific transmembrane cognate receptor (gp80 or IL-6Rα), which triggers the homodimerization of gp130 (IL-6Rβ) and leads to the activation of the Jak/Stat signaling pathway, particularly of STAT-3 (6). In addition to its membrane bound form, the IL-6R is also found in a soluble form (sIL-6R), which when complexed with IL-6, is capable of stimulating cells via interaction with gp130. Importantly then, IL-6/sIL-6R complexes are capable of acting as an agonist on cell types that, although they express gp130, would not inherently respond to IL-6 alone. Hyper-IL-6 (HIL-6) is a superagonistic designer cytokine consisting of the human IL-6 linked by a flexible peptide chain to sIL-6R (7). HIL-6 is fully active on gp130-expressing cells at concentrations 100 to 1000 fold lower than unlinked IL-6/sIL-6R, and exhibits a super agonistic effect both in vitro and in vivo, due in part to its prolonged half-life (8).

The development of a recombinant adenoviral vector, Ad.HIL6gfp, encoding a HIL-6 gene under the transcriptional control of the CMV immediate early promoter in an E1A/E3 deleted, replication defective adenoviral vector backbone has been previously reported (9). It was shown that gene therapy with the construct encoding HIL-6 reversed fulminant hepatic failure (9). Fulminant hepatic liver failure is a catastrophic condition caused by massive hepatocellular apoptosis and necrosis. Inhibition of hepatocyte apoptosis and the enhancement of the endogenous potential for liver regeneration could potentially form an effective basis for treatment of this condition. In response to injury in the liver, IL-6 mediates the acute-phase response and induces both cytoprotective and mitogenic functions. In a mouse model of acute liver failure induced by D-galactosamine administration, a single low dose of an adenoviral-hyper IL-6 vector, in contrast to an adeno-IL-6 vector, maintained liver function, prevented the progression of liver necrosis, and induced liver regeneration, leading to dramatically enhanced survival. It was concluded that HIL-6 gene therapy may be useful for the treatment of fulminant hepatic failure, which is often fatal even following treatment by transplantation (9).

U.S. Pat. No. 5,462,731 discloses the use of IL-6 in the treatment of chronic lymphocytic leukemia and B-cell lymphomas. It also discloses the use of IL-6 and IL-6R in the treatment of these diseases. U.S. Pat. No. 5,902,576 discloses an anti-tumor pharmaceutical composition, which includes cells into which a gene encoding human IL-6 has been inserted.

U.S. Pat. No. 5,919,763 discloses a method for treating a liver injury in a subject comprising administering to the subject an IL-6/sIL-6R complex. U.S. Pat. No. 5,919,763 particularly describes the efficacy of Hyper-IL-6 to accelerate liver regeneration as detected by liver proliferation and reconstitution of liver weight. International Patent Application WO 99/62534 further discloses a gene therapy method for treating an injury of a liver in a subject comprising administering to the subject a vector carrying Hyper-IL-6 chimera gene.

International Patent Application WO 99/02552 discloses the production of fusion proteins (chimeras) comprising the naturally occurring form of sIL-6R and IL-6 in mammalian expression systems. The IL-6/sIL-6R fusion proteins thus produced exert potent activity on tumor cells, which are usually non-responsive to IL-6 or sIL-6R alone. In addition, the IL-6/sIL-6R chimeras are shown to be highly effective in ensuring the success of engraftment of human bone marrow transplanted cells.

U.S. Pat. No. 6,475,755 describes a recombinant defective adenovirus vector, which encodes an IL-6 antagonist. The recombinant adenovirus vector is shown to inhibit IL-6 activity when transduced to cells in vitro and when transduced to mice in vivo. U.S. Pat. No. 6,475,755 thus claims a method for inhibiting the activity of IL-6 in human cells in vitro using the recombinant adenovirus vector encoding the IL-6 antagonist.

A method for treating tumors using recombinant adenoviral vectors encoding a cytokine is disclosed, for example in U.S. Pat. No. 6,066,624, wherein the recombinant adenoviral vector encoding the cytokine constitutes a “combination gene therapy” as it is co-administered with a recombinant adenoviral vector encoding a suicide protein. Additional studies describe the anti-tumor effect of adenoviral vectors encoding IL-6 (10, 11).

However, the prior art lacks an efficient method for treating cancer using E1A deleted adenoviral vectors encoding a cytokine together with its cognate receptor.

### SUMMARY OF THE INVENTION

According to a first aspect, the present invention provides a method for treating a neoplastic disease in a subject comprising administering to the subject a therapeutically effective amount of a recombinant replication defective adenovirus vector exerting oncolytic activity, said vector comprising a DNA sequence encoding an IL-6/soluble IL-6 receptor (sIL-6R) complex or a biologically active variant thereof, said DNA sequence being operably linked to a promoter. The recombinant adenovirus vector is selected from the group of adenoviruses that have inoperable, mutated or deleted E1A. Currently preferred are adenoviruses comprising partially or fully deleted E1 adenovirus vectors, partially or fully deleted E3 adenovirus vectors, partially or fully deleted E4 adenoviral vectors, and combinations thereof. However, further deletions of wild-type genes in the adenovirus genome are encompassed in the present invention so long as the virus retains its replication and transduction capabilities. Preferably, the adenovirus serotype 2 and 5 are used as a source for adenovirus vector.

The term “oncolytic activity” as used herein refers to cytotoxic effects in vitro and/or in vivo exerted on tumor cells without any appreciable or significant deleterious effect to normal cells under the same conditions.

In a preferred embodiment, the IL-6/sIL-6R complex is hyper-IL-6.

In another embodiment of the present invention, the recombinant adenovirus vector further comprising at least one other non-viral protein. Preferably, the non-viral protein enhances gp130 signaling or the down-stream molecular events resulting from gp130 signaling. More preferably, the non-viral protein is selected from STAT-3 and NF-IL6.

In a further embodiment, the non-viral protein is selected from the group of cytokines, suicide factors, transcription factors, and biologically active variants thereof. Preferably, the non-viral protein is a cytokine having a receptor complex comprising gp130.

In a further embodiment, the present invention also relates to a method for treating a neoplastic disease in a subject comprising administering a therapeutically effective amount of a first recombinant replication defective adenovirus vector comprising a DNA sequence encoding IL-6 or a biologically active variant thereof operably linked to a promoter; and substantially at the same time administering a second recombinant replication defective adenovirus vector, said second vector comprising a DNA sequence encoding sIL-6R or a biologically active variant thereof operably linked to a promoter, wherein said first and second vectors exert oncolytic activity.

In another embodiment, the present invention provides a method for treating a neoplastic disease in a subject comprising administering to the subject a therapeutically effective amount of transfected eukaryotic cells, said transfected cells being transduced with a recombinant replication defective adenovirus vector exerting oncolytic activity, said vector comprising a DNA sequence encoding an IL-6/sIL-6R complex or a biologically active variant thereof, said DNA sequence is operably linked to a promoter.

The neoplastic diseases that may be treated by the methods of the invention include, but are not limited to, benign solid tumors, malignant solid tumors, benign proliferative diseases of the blood, and malignant proliferative diseases of the blood. Preferably, liver cancer may be treated by the method of the invention.

The vectors of the invention may be administered through any suitable route. One particularly preferred route of administration is direct injection into the tumor or in proximity to the tumor mass.

In another aspect, the present invention provides a recombinant replication defective adenovirus vector exerting oncolytic activity, said vector selected from the group consisting of partially or fully deleted E1 adenovirus vectors, partially or fully deleted E3 adenovirus vectors, partially or fully deleted E4 adenovirus vectors, or combinations thereof, said vector further comprising a DNA sequence encoding an IL-6/soluble IL-6 receptor (sIL-6R) complex or a biologically active variant thereof operably linked to a promoter.

It is now disclosed for the first time that unexpectedly the adenoviral vectors that are E1and E3 deleted comprising sequences encoding IL-6/sIL-6R complex under a CMV promoter achieve significantly higher yields, as compared to previously known adenoviral vectors encoding IL-6 or IL-6/sIL-6R complex, thereby greatly enhancing the ability to obtain viral stocks on a scale suitable for therapeutic uses in general and in humans in particular.

Thus, in a preferred embodiment of the present invention, the recombinant adenovirus vector is Ad.HIL6gfp. The Ad.HIL6gfp adenoviral vector has deleted E1 and E3 genes comprising a DNA sequence encoding HIL-6 under a CMV promoter. Clearly the gfp reporter gene construct is used for laboratory experimentation, however it is not necessary to use any reporter gene in constructs designed for therapeutic uses.

In a further aspect, the present invention provides a pharmaceutical composition for treating a neoplastic disease comprising as an active ingredient a recombinant adenovirus vector exerting oncolytic activity, said vector comprising a DNA sequence encoding an IL-6/soluble IL-6 receptor (sIL-6R) complex or a biologically active variant thereof selected from the group consisting of partially or fully deleted E1 adenovirus vectors, partially or fully deleted E3 adenovirus vectors, partially or fully deleted E4 adenovirus vectors, or combinations thereof.

These and other embodiments of the present invention will be better understood in relation to the figures, description, examples, and claims that follow.

### BRIEF DESCRIPTION OF THE DRAWINGS

FIG. 1 shows a schematic diagram of the recombinant adenoviral vector Ad.HIL6gfp encoding the CMV immediate early enhancer/promoter (CMV), a chimeric intron (I), and a bicistronic gene containing the 1.6-kb hyper-IL-6 (HIL-6) cDNA, followed by the polio virus IRES (ir) and an enhanced green fluorescence protein gene (gfp), followed by the late SV40 polyadenylation site in an E1/E3-deleted Ad5 backbone derived from the adenoviral vector pAdeasy-1 (9).

FIG. 2a-b shows that the recombinant adenoviral vector Ad.HIL6gfp replicates and induces cytopathic effects in transduced tumor cell lines. Immortalized and tumor derived cell lines of human origin were transduced with E1/E3 deleted adenoviral vectors, Ad.HIL6gfp, Ad.IL6gfp, or Ad.gpf, in two sequential rounds of viral infection (Infection Cycle). Cell lines tested included: FIG. 2a shows HEK 293 transduced cells. FIG. 2b shows HepG2 transduced cells. All viruses contained a gene encoding the green fluorescence protein (gfp), which can be detected by fluorescence microscopy. Cytopathic effects (CPE) were detected by light microscopy. In the initial cycle of infection (1°), cells were exposed to the viruses at a multiplicity of infection (m.o.i.)˜1. Subsequent rounds of infection (2°) were performed with 10% of the crude lysate from the preceding infection cycle (Original magnification×100).

FIG. 3 shows the cytopathic effects of Ad.HIL6gfp infection in HepG2 cells. HepG2 cells plated in 96 well microtiter dishes were exposed to either Ad.gfp or Ad.HIL6gfp at m.o.i˜1. Cultures were incubated for 2 days to enable virus propagation and CPE formation, and then stained with methylene blue. Cell survival was calculated as the ratio of bound methylene blue stain in viral infected cultures compared to identical wells of mock-infected cells.

FIG. 4a-b shows that HIL-6 protein enhances replication of E1A deficient Ad5 vector in HepG2 cells. FIG. 4a shows GH329 cells. FIG. 4b shows HepG2 cells. GH329 and HepG2 cells were infected with Ad.gfp at m.o.i. 10, in the presence (+), or absence (−) of HIL-6 protein (2 ng/ml). Virus particles harvested from the initial infection cycle (1°) of the indicated cell type were subsequently applied to GH329 cells in a second cycle of infection (2°) in order to estimate viral yield. Cultures were photographed following 3 days of incubation using an inverted fluorescent microscope. Original magnification×100, or ×40 where indicated.

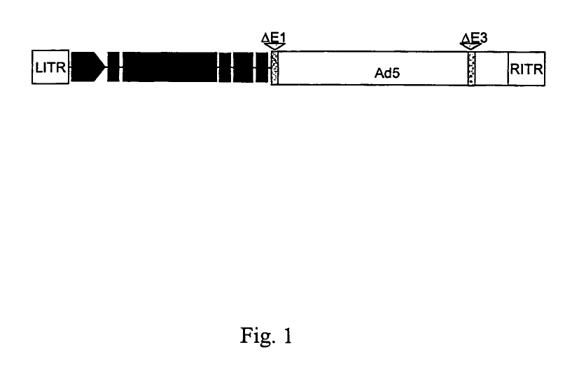

### DETAILED DESCRIPTION OF THE INVENTION

The present invention is based in part on the previous finding of Spergel and Chen-Kiang that exogenous administration of IL-6 brings about replication of an E1 deleted adenovirus in HepG2 hepatoblastoma cells (3).

The present invention thus relates to exogenous introduction of an IL-6/sIL-6R complex to tumor cells through an adenoviral vector. According to the present invention, introduction of the adenoviral vector encoding IL-6/sIL-6R complex, particularly hyper-IL-6 (HIL-6), kills tumor cells, preferably those cells expressing gp130, even in the absence of E1A complementation, or without any addition of either exogenous IL-6or sIL-6R protein.

In describing the present invention, the following terms will be employed, and are intended to be defined as indicated below.

The term “adenovirus vector” means a vector derived from adenovirus, including without limitation serotype 1, 2, 5 or 6. Adenovirus vectors can have one or more of the adenovirus wild-type genes deleted in whole or part, but retain functional flanking inverted terminal repeat (ITR) sequences. Functional ITR sequences are necessary for the rescue, replication and packaging of the adenovirus virion. Thus, an adenovirus vector is defined herein to include at least those sequences required for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging. Typically, the ITRs are flanking a restriction site that can be used for subcloning of the transgene, either directly using the restriction site available, or by excision of the transgene with restriction enzymes followed by blunting of the ends, ligation of appropriate DNA linkers, restriction digestion, and ligation into the site between the ITRs.

The present invention provides a recombinant replication defective adenovirus vector exerting oncolytic activity, said vector selected from the group consisting of partially or fully deleted E3 adenovirus vectors, partially or fully deleted E3 adenovirus vectors, partially or fully deleted E4 adenovirus vectors, or combinations thereof, said vector further comprising a DNA sequence encoding an IL-6/soluble IL-6receptor (sIL-6R). complex or a biologically active variant thereof, operably linked to a promoter.

It is now disclosed for the first time that unexpectedly the viral vectors that are E3 and E3 deleted comprising IL-6/sIL-6R complex under a CMV promoter achieve enhanced yields compared to known adenoviral vectors encoding IL-6 or IL-6/sIL-6R complex thereby greatly boosting the ability to obtain viral stocks on a scale suitable for therapeutic uses in general and in humans in particular. Thus, in a preferred embodiment of the invention, the recombinant adenoviral vector is Ad.HIL6gfp as described in the Examples below.

It should be kept in mind that the E3 gene includes both the E1A and E1B DNA sequences. An adenovirus vector partially or fully deleted of the DNA sequences encoding the E3 gene and/or the E3 gene such as, for example, E1A deleted and/or E2B deleted, is contemplated in the application. Also provided by this invention is an adenovirus vector having more extensive deletions such as, for example, of a non-essential DNA sequence in adenovirus early region 4, or gutless adenoviral vectors. Thus, the present invention encompasses insertion, deletion, or substitution of nucleotides in E3 , E3 , and E4 DNA sequences and/or in any other wild-type adenovirus genes so long as the sequences provide for functional rescue, replication, and packing. The adenovirus vectors in the present invention having tropism associated with infection of humans. However, adenovirus vectors having tropism to other mammals or to any animal are also contemplated in the invention.

The term “recombinant adenovirus vector” as used herein defines a recombinant adenovirus vector comprising: (a) the DNA of, or corresponding to, at least a portion of the genome of an adenovirus which portion is capable of transducing into a target cell at least one selected gene and is capable of promoting replication and packaging; and (b) at least one selected gene (or transgene) operatively linked to regulatory sequences directing its expression, the gene flanked by the DNA of (a) and capable of expression in the target cell in vivo or in vitro. Thus, when refer to a “recombinant adenovirus” it is meant the adenovirus that has been genetically altered, e.g., by the addition or insertion of a selected gene.

A “gene” or a “sequence which encodes” a particular protein, is a nucleic acid molecule which is transcribed (in the case of DNA) and translated (in the case of mRNA) into a polypeptide in vitro or in vivo when placed under the control of appropriate regulatory sequences. The boundaries of the gene are determined by a start codon at the 5′ (amino) terminus and a translation stop codon at the 3′ (carboxy) terminus. A gene can include, but is not limited to, cDNA from eukaryotic mRNA, genomic DNA sequences from eukaryotic DNA, and even synthetic DNA sequences. A transcription termination sequence will usually be located 3′ to the gene sequence. Typically, polyadenylation signal is provided to terminate transcription of genes inserted into a recombinant virus.

As is known to those of skill in the art, the term “polypeptide” or “protein” means a linear polymer of amino acids joined in a specific sequence by peptide bonds. As used herein, the term “amino acid” refers to either the D or L stereoisomer form of the amino acid, unless otherwise specifically designated.

The term “transgene” refers to a particular nucleic acid sequence encoding a polypeptide or a portion of a polypeptide to be expressed in a cell into which the nucleic acid sequence is inserted. The term “transgene” is meant to include (1) a nucleic acid sequence that is not naturally found in the cell (i.e., a heterologous nucleic acid sequence); (2) a nucleic acid sequence that is a mutant form of a nucleic acid sequence naturally found in the cell into which it has been inserted; (3) a nucleic acid sequence that serves to add additional copies of the same (i.e., homologous) or a similar nucleic acid sequence naturally occurring in the cell into which it has been inserted; or (4) a silent naturally occurring or homologous nucleic acid sequence whose expression is induced in the cell into which it has been inserted. By “mutant form” is meant a nucleic acid sequence that contains one or more nucleotides that are different from the wild-type or naturally occurring sequence, i.e., the mutant nucleic acid sequence contains one or more nucleotide substitutions, deletions, and/or insertions. In some cases, the transgene may also include a sequence encoding a leader peptide or signal sequence such that the transgene product will be secreted from the cell.

In the present invention, the transgene is IL-6, sIL-6R, IL-6/sIL-6R complex, or a biologically active variant thereof. Most preferably, the IL-6/sIL-6R complex is hyper-IL-6(HIL-6). The present invention encompasses IL-6/sIL-6R complex in any order.

Hereinafter, the term “IL-6/sIL-6R complex” refers both to a bimolecular protein complex which features both the IL-6 protein and the soluble IL-6 receptor protein, designated sIL-6R, and to a unimolecular protein which includes the bioactive portions of IL-6and sIL-6R connected with a flexible linker, substantially as previously described in International Patent Applications WO 97/32891 and WO 99/62534 and in (7, 9), incorporated by reference as if fully set forth herein, as well as any biologically active equivalents thereof.

The bimolecular protein complex includes both IL-6 and sIL-6R at any order as well as biologically active variants thereof. The term “biologically active variants” includes any homologous polypeptide to either IL-6, sIL-6R, or to any other protein of the present invention, which includes any amino acid substitution, deletion, or addition, which still maintains the biological activity of the original polypeptide. Thus, a biologically active variant of an IL-6/sIL-6R complex retains the capability to directly stimulate the membrane receptor for the IL-6/sIL-6R complex known as gp130 or any other component of the down-stream signaling of gp130.

A variation of the unimolecular protein, which includes amino acids 114-323 of the sIL-6R-polypeptide, is also included. A variation, which includes amino acids 113-323 of the sIL-6R-polypeptide and amino acids 29-212 of the IL-6-polypeptide is also encompassed in the present invention. Other variations and combinations as disclosed in PCT No. WO 97/32891 are also included in the unimolecular protein embodiment of the IL-6/sIL-6R complex so long as the complex retains its capability to stimulate the gp130 or any component of the down-stream signaling of gp130.

The term “operably linked” refers to the arrangement of various nucleic acid molecule elements relative to each other such that the elements are functionally connected and are able to interact with each other. Such elements may include, without limitation, a promoter, an enhancer, a polyadenylation sequence, one or more introns and/or exons, and a coding sequence of a gene of interest to be expressed (i.e., the transgene). The nucleic acid sequence elements, when operably linked, act together to modulate the activity of one another, and ultimately may affect the level of expression of the transgene. By modulate is meant increasing, decreasing, or maintaining the level of activity of a particular element. Typically, transduction of the transgene of the invention increases the expression of the transgene, preferably that of IL-6/sIL-6R complex. The position of each element relative to other elements may be expressed in terms of the 5′ terminus and the 3′ terminus of each element.

The term “promoter” refers to a nucleic acid sequence that regulates, either directly or indirectly, the transcription of a corresponding nucleic acid coding sequence to which it is operably linked. The promoter may function alone to regulate transcription, or, in some cases, may act in concert with one or more other regulatory sequences such as an enhancer or silencer to regulate transcription of the transgene. The promoter comprises a DNA regulatory sequence, wherein the regulatory sequence is derived from a gene, which is capable of binding RNA polymerase and initiating transcription of a downstream (3′-direction) coding sequence.

Recombinant adenoviruses containing IL-6, sIL-6R, IL-6/sIL-6R complex, or any other transgenes of the invention can be driven by the E1A promoter, the E3 promoter, other endogenous adenoviral promoters, or by any heterologous promoter including, but not limited to, the cytomegalovirus promoter, Rous Sarcoma Virus-Long Terminal Repeat (LTR), murine leukemia virus LTR, simian virus 40 early and late promoters, and herpes simplex virus thymidine kinase. In addition, adenoviral vectors in which the E3 and/or E3 genes are driven by a heterologous promoter such as, for example, adenoviral vector with a PSA promoter driven E1A, are also contemplated in the present invention.

Inducible promoters can also be used in the adenoviral vector of this invention. These promoters will initiate transcription only in the presence of an additional molecule. Examples of inducible promoters include, but not limited to, those obtainable from a heat shock gene, a metallothionine gene, or those obtainable from steroid hormone-responsive genes. Tissue specific expression has been well characterized in the field of gene expression and tissue specific and inducible promoters are very well known in the art. These promoters are used to regulate the expression of the foreign gene after it has been introduced into the target cell. Thus, the present invention contemplates a constitutive promoter, a promoter enabling tissue specific expression of the transgene, a promoter controlled by the administration of exogenous substances, or a promoter regulated in a fashion combining one or more of these features.

Exogenous substances that may be administered to regulate promoter function are selected from the group consisting of natural substances, synthetic substances, synthetic antibiotic drugs, and natural antibiotic drugs.

The term “transduction” denotes the delivery of a DNA molecule to a recipient cell either in vivo or in vitro, via a replication-defective viral vector, such as via a recombinant adenovirus.

The term “transfection” is used to refer to the uptake of foreign DNA by a mammalian cell. A cell has been “transfected” when exogenous DNA has been introduced inside the cell membrane. A number of transfection techniques are known in the art. See, Graham et al. (1973) Virology, 52:456; and Sambrook et al. (1989) Molecular Cloning, a laboratory manual, Cold Spring Harbor Laboratories, New York. Such techniques can be used to introduce one or more exogenous DNA moieties, such as a viral vector and other nucleic acid molecules, into suitable host cells. The term refers to both stable and transient uptake of the genetic material.

The vectors of the present invention may be useful for the introduction of additional genes in gene therapy. Thus, for example, the adenoviral vector of this invention can contain an additional exogenous gene for the expression of a protein effective in regulating the cell cycle, such as p53, Rb, or mitosin, or a biologically active variant thereof, or in inducing cell death, such as the conditional suicide gene thymidine kinase, the latter must be used in conjunction with a thymidine kinase metabolite in order to be effective, or any other anti-tumor gene, such as for example a toxin.

The present invention also encompasses recombinant adenoviruses engineered to alter viral binding and entry through cell receptors, other than those native to adenoviruses. In addition, adenoviruses comprising combinations of transgenes, namely IL-6, sIL-6R, or an IL-6/sIL-6R complex gene with other genes encoding non-viral proteins including, but not limited to, suicide genes and genes encoding cytokines, transcription factors, tumor suppressors, apoptosis inducers, inhibitors of cyclin-dependent kinases, variants thereof, and the like are contemplated in the invention. Additionally, adenoviral vectors encoding proteins that enhance gp130 signaling or any down-stream molecular events resulting from gp130 signaling such as, for example STAT-3 and/or NF-IL-6, are contemplated in the invention.

The present invention further encompasses adenoviral vectors comprising genes of cytokines the receptor complex of which contains gp130; adenoviral vectors encoding in addition to IL-6, sIL-6R, or IL-6/sIL-6R complex or a variant thereof an integrin binding site to enhance viral infectivity; and combinations of vectors whereby the gene encoding IL-6, sIL-6R, or IL-6/sIL-6R complex is administered through a vector (other than adenovirus) and applied alone, or in combination with recombinant adenovirus vectors of the invention. Examples of recombinant viral vectors (other than adenovirus) that may encode IL-6, sIL-6R, or IL-6/sIL-6R complex or a biologically active variants thereof are adeno-associated vectors, Herpes Virus, Measles Virus, Newcastle Disease virus vectors, or any other viral vector exerting oncolytic activity.

The present invention further encompasses hybrid vectors, wherein the ITR sequences of the recombinant adenovirus are replaced with ITR sequences derived from other viruses exemplified, but not limited to, adeno-associated virus (AAV), Epstein-Barr virus, and retroviruses.

The adenoviral vectors of this invention may also contain a selectable reporter permitting evaluating the transfection yield such as, for example, green fluorescent protein (gfp). It should be noted that the reporter is used for laboratory experimentation, however it is not necessary to use any reporter gene in constructs designed for therapeutic uses.

The present invention provides a method for treating a neoplastic disease in a subject, the subject being an animal or human, comprising administering to the subject a therapeutically effective amount of a recombinant replication defective adenovirus vector exerting oncolytic activity, said vector comprising a DNA sequence encoding an IL-6/sIL-6R complex or a biologically active variant thereof, said DNA is operably linked to a promoter. Preferably, the IL-6/sIL-6R complex is hyper-IL-6 (HIL-6). More preferably, the adenoviral vector is Ad.HIL6.gfp.

As used hereafter, the terms “neoplasm” and “neoplastic” refer to a tumor and/or to an abnormal tissue, including metastatic disease, that grows by cellular proliferation more rapidly than normal, continues to grow after the stimuli that initiated the new growth cease, shows partial or complete lack of structural organization and functional coordination with normal tissue, and usually forms a distinct mass of tissue which may be either benign or malignant.

In another embodiment, the present invention provides a method for treating a neoplastic disease in a subject comprising administering a therapeutically effective amount of a first recombinant replication defective adenoviral vector comprising a DNA sequence encoding IL-6 or a biologically active variant thereof operably linked to a promoter; and substantially at the same time administering a second recombinant replication defective adenoviral vector, said second vector comprising a DNA sequence encoding sIL-6R or a biologically active variant thereof operably linked to a promoter, wherein said first and second vectors exert oncolytic activity.

Further provided by this invention is a method for treating a neoplastic disease comprising a step of administering to a tumor a transfected eukaryotic host cell, for example an animal cell or mammalian cell, having inserted a recombinant replication defective adenovirus vector described above. Methods of transducing host cells with adenoviral vectors are known in the art, and include, but are not limited to, transfection, electroporation, and microinjection. Transfection may be performed in the presence of cationic lipids or polymers using calcium phosphate, thus bypassing normal receptor-mediated entry of adenovirus.

A wide variety of neoplastic diseases can be treated by the same therapeutic strategy of the present invention. Neoplastic diseases include, but are not limited to, benign solid tumors, malignant solid tumors, benign proliferative diseases of the blood, and malignant proliferative diseases of the blood. Representative examples include colon carcinoma, prostate cancer, breast cancer, lung cancer, skin cancer, liver cancer, bone cancer, ovary cancer, pancreas cancer, brain cancer, head and neck cancer, and lymphoma.

As used throughout this application, the term animal is intended to be synonymous with mammal and is to include, but not be limited to, bovine, porcine, feline, simian, canine, equine, murine, rat or human. However, adenovirus vectors derived from avian species are also encompassed in the present invention. Host cells include, but are not limited to, any neoplastic or tumor cell, such as osteosarcoma, ovarian carcinoma, breast carcinoma, melanoma, hepatocarcinoma, lung cancer, brain cancer, colorectal cancer, hematopoietic cell, prostate cancer, cervical carcinoma, retinoblastoma, esophageal carcinoma, bladder cancer, neuroblastoma, or renal cancer.

Any of the recombinant adenovirus vectors and transformed host cells described herein is useful as compositions for therapy. The transformed host cells may be transplanted to the same or other animal from which they were derived. When used pharmaceutically, they can be combined with various pharmaceutically acceptable carriers. Suitable pharmaceutically acceptable carriers are well known to those of skill in the art and are exemplified below. The compositions can then be administered therapeutically or prophylactically, in effective amounts, described in more detail below.

As used herein, the term “therapeutically effective amount” is intended to mean the amount of vector or of transformed cells, which exerts oncolytic activity, causing attenuation or inhibition of tumor cell proliferation leading to tumor regression. For example, one dose of adenovirus vector may contain from about 10⁷ to about 10¹³ infectious units. An effective amount will vary on the pathology or condition to be treated, by the patient and his status, and other factors well known to those of skill in the art. Effective amounts are easily determined by those of skill in the art.

The term “oncolytic activity” as used herein refers to cytotoxic effects in vitro and/or in vivo exerted on tumor cells without any appreciable or significant deleterious effects to normal cells under the same conditions. The cytotoxic effects under in vitro conditions are detected by various means as known in prior art, for example, by staining with a selective stain for dead cells, by inhibition of DNA synthesis, or by apoptosis. Detection of the cytotoxic effects under in vivo conditions is performed by methods known in the art and exemplified in Example 4 below.

Methods of treating a neoplastic disease may include administration of the compounds of the present invention as a single active agent, or in combination with additional methods of treatment including, but not limited to, irradiation therapy, therapy with immunosuppressive agents, chemotherapeutic or anti-proliferative agents, including cytokines. The methods of treatment of the invention may be in parallel to, prior to, or following additional methods of treatment.

Any of the vectors described herein are useful for the treatment of a neoplastic disease. When used pharmaceutically, the vectors of the invention can be combined with one or more pharmaceutically acceptable carriers. Pharmaceutically acceptable carriers are well known in the art and include aqueous solutions such as physiologically buffered saline or other solvents or vehicles such as glycols, glycerol, vegetable oils (e.g., olive oil) or injectable organic esters. A pharmaceutically acceptable carrier can be used to administer the compositions of the invention to a cell in vitro or to a subject in vivo.

A pharmaceutically acceptable carrier can contain a physiologically acceptable compound that acts, for example, to stabilize the composition or to increase the absorption of the agent. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents or preservatives, which are particularly useful for preventing the growth or action of microorganisms. Various preservatives are well known and include, for example, phenol and ascorbic acid. One skilled in the art would know that the choice of a pharmaceutically acceptable carrier, including a physiologically acceptable compound, depends, for example, on the route of administration of the polypeptide. For example, a physiologically acceptable compound such as aluminum monosterate or gelatin is particularly useful as a delaying agent, which prolongs the rate of absorption of a pharmaceutical composition administered to a subject. Further examples of carriers, stabilizers or adjutants can be found in Martin, Remington's Pharm. Sci., 15th Ed. (Mack Publ. Co., Easton, 1975), incorporated herein by reference.

As used herein, “pharmaceutical composition” or “composition” refers to any of the compositions of matter described herein. The compositions can then be administered therapeutically or prophylactically. They can be contacted with the host cell in vivo, ex vivo, or in vitro, in a therapeutically effective amount. In vitro and in vivo means of transfecting the vectors of the invention are provided below.

According to the invention, any suitable route of administration of the vectors may be adapted, including but not limited to, intravenous, oral, buccal, intranasal, inhalation, topical application to a mucosal membrane or injection, including intratumoral, intradermal, intrathecal, intracisternal, intralesional or any other type of injection. Administration can be effected continuously or intermittently and will vary with the subject and the condition to be treated.

According to the invention, the Ad.HIL6 vector exhibits unique qualities by means of endowing the infected cells with a complete IL-6 autocrine arc enabling replication of the virus in tumor cells in which replication would otherwise be restricted. Without wishing to be limited to a single hypothesis, this virus may be able to spread from the primary infected tumor cells to neighboring tumor cells and, as such, may be more useful as a selective oncolytic agent in attacking and killing tumor cells in patients. Furthermore, due to the pro-inflammatory nature of IL-6 , expression of HIL-6 by the tumor cells may also enhance attraction of immune cells to the site of the tumor and thus increase immune-mediated tumor rejection.

The principles and operation of the methods of treatment which feature an oncolytic recombinant adenoviral vector encoding an IL-6/sIL-6R complex, particularly HIL-6, according to the present invention, may be better understood with reference to the non-limiting illustrative examples below. These examples describe certain experiments performed with the constructs of the present invention, demonstrating the efficacy of the viral construct in a biological system.

### EXAMPLES

Materials and Methods

Cells

Human cell lines, including human embryonic kidney HEK 293 cells (12), GH329 cells (13) the human hepatocellular carcinoma derived cell lines HepG2 (14) and HUH-7 (15), and immortalized adult human kidney HK cells were cultured in Dulbeco's Modified Eagle's Medium (DMEM) supplemented with 10% fetal calf serum and 2 mM glutamine, and kept at 37° C. in a humidified incubator containing 5% CO₂.

Recombinant Adenoviral Vectors

The recombinant adenoviral vectors Ad.HIL6gfp, Ad.IL6gfp and Ad.gfp were constructed using the AdEasy-1 system (16), which is based on the E1/E3 deficient, human, adenovirus type 5. Ad.HIL6gfp encoding the IL-6/sIL-6R fusion protein (also designated complex) was constructed using a human hyper-IL-6 cDNA gene coding the human sIL-6R (amino acid residues 1-323) and human IL-6 (amino acid residues 29-212) fused by a synthetic DNA linker coding for the amino acid sequence Arg-Gly-Gly-Gly-Ser-Gly-Gly-Gly-Gly-Ser-Val-Glu (9). In order to allow convenient monitoring of adenoviral transduction and gene expression, a bicistronic hyper-IL-6/IRES (poliovirus)/gfp gene construct was made by ligation of a SnaBI-Not I DNA fragment containing a portion of the CMV promoter and the hyper-IL-6 gene derived from the expression plasmid pCI-HIL6 to the same sites in the plasmid pGEMIRESegfp (a kind gift from W. Lindenmaier, G B F, Braunschweig, Germany). To prepare the Ad.HIL6gfp virus construct, an Ac1 I—Nru I DNA fragment from pGEMhil6IRESgfp containing the CMV promoter driven HIL6gfp gene construct was cloned into the Eco RV site of pShuttle and subsequently introduced into the pAdEasy-1 vector by homologous recombination in BJ5183 cells. Ad.HIL6gfp was rescued from pAdEasy-HIL6gfp by transfection in 293 cells and plaque purified twice by limiting dilution. The Ad.IL6gfp virus was constructed in a similar manner using the human IL-6cDNA.

Vector mediated transgene expression was confirmed by analysis of conditioned media from adenoviral vector transduced 293 or HUH-7cells on BAF/3/gp130 and BAF/3/gp130/IL-6R indicator cell lines, which are dependent on hyper-IL-6 and IL-6, respectively, for cell proliferation. The control virus, Ad.gfp, was kindly provided by H. Giladi (Hadassah Medical Organization) and was constructed by homologous recombination of pAdEasy-1 with the pAd-Track shuttle vector (16). For large-scale adenoviral preparations, HEK 293 cells cultured on 15 cm dishes in DMEM supplemented with 10% fetal calf serum and 2 mM glutamine, were infected at a multiplicity of infection of 1-5 and grown for 2-3 days. The infected cells were then collected by centrifugation (2000×g for 5 minutes) and viruses harvested by gentle lysis (3 cycles of freeze/thawing) of cells in a solution of PBS containing 0.68 mM CaCl₂, 0.50 mM MgCl₂ and 10% glycerol. The cell extracts were then clarified by centrifugation at 10,000×g for 4 minutes at room temperature and stored at −80° C. for use in animal studies. Viral titers were determined by infection of HEK 293 cells cultured in 24 well dishes, and scored 2-3 days after infection by visualization of the gfp marker using an inverted fluorescence microscope.

Viral Replication and Cytopathic Effects

Viral transduction in human (E1A⁻) tumor and/or immortalized cell lines, transduced with recombinant adenoviruses (Ad.HIL6gfp, Ad.IL6gfp, or Ad.gfp) was monitored for gfp reporter gene expression by fluorescence microscopy. Clearly this reporter gene construct is used for laboratory experimentation, however it is not necessary to use any reporter gene in constructs designed for therapeutic uses. Cytopathic effects (CPE) resulting from the viral infection/transduction was monitored three to seven days following post-infection by visual examination using an phase-contrast microscope. Alternatively, cell survival was measured by the methylene blue staining as described by Oliver M. H., et a. (1989) J. Cell Science 92, 513-518. Briefly, cell cultures plated in 96 well tissue culture dishes were fixed by treatment with a solution of 2% Formaldehyde and 0.2% gluteraldehyde. The fixation solution was removed and the cells were stained with a solution of 1% Methylene Blue in 0.01 M borate buffer (pH 8.5). After 30 minutes, the excess die was removed and the wells were washed 4 times by submersion in distilled H₂O. Bound die was then eluted with 100 μl 0.1 M HCl and absorbance at 650 nm (A₆₅₀) was monitored using a microplate photometer.

Recombinant HIL-6 Protein

Recombinant HIL-6 protein was prepared from culture supernatants of genetically engineered Chinese Hamster Ovary (CHO) cells carrying a HIL-6 gene cassette. HIL-6 was purified from supernatants by anion-exchange chromatography and gel filtrations, and then visualized by sodium dodecyl sulfate-polyacrylamide gel electrophoresis and silver staining (8).

### Example 1

### An Adenoviral Vector Encoding HIL-6 Replicates and Induces Cytopathic Effects in Transduced Tumor Cell Lines

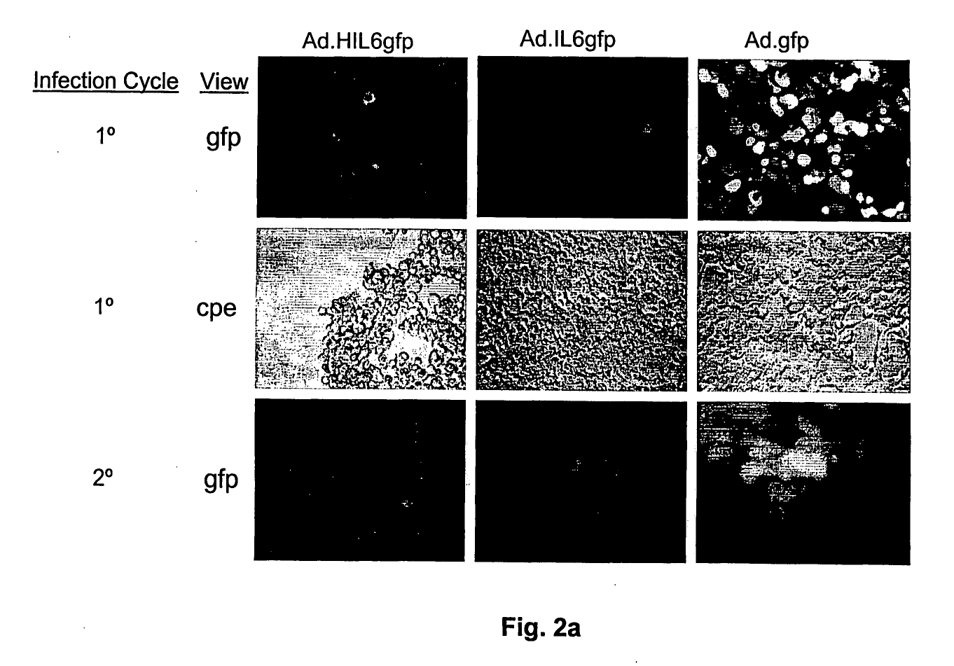

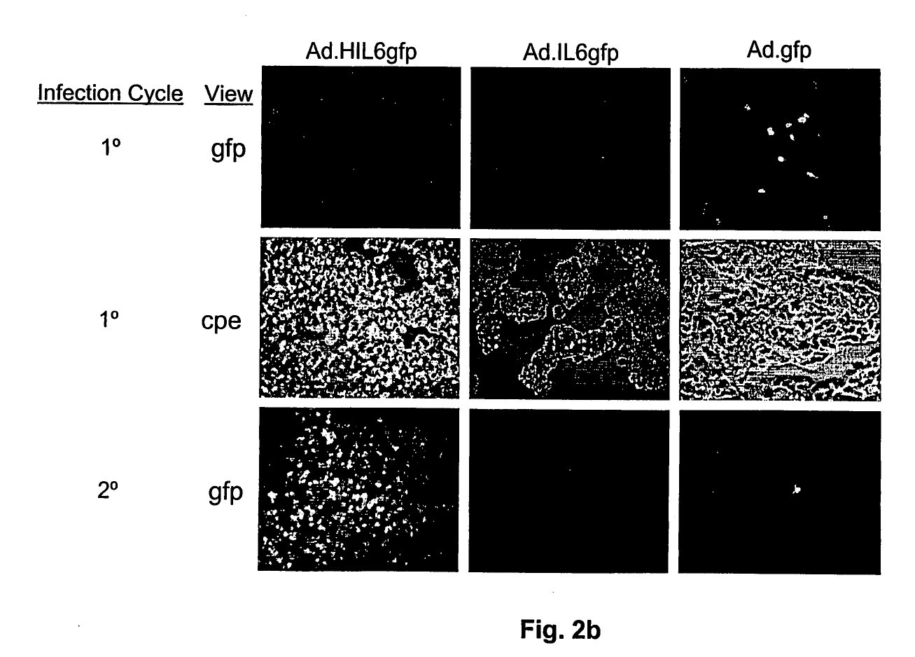

In order to explore whether expression of a virally encoded HIL-6 gene would enable E1A deficient adenoviral vectors to replicate in otherwise non-permissive cell lines, the ability of a series of E1/E3 deficient adenoviral vectors that encoded either a human HIL-6 gene (Ad.HIL6gfp), a human IL-6cDNA (Ad.IL6gfp), or no cytokine transgene (Ad.gfp) to infect and replicate in immortalized or tumor derived cell lines was determined. The cell lines examined included the human hepatocellular carcinoma derived cell lines, HepG2, and E1A positive HEK 293 cells. All recombinant viruses used for these experimental systems encoded the gfp reporter gene to enable efficient verification of viral transduction. As shown in FIG. 2a, all of the recombinant viruses showed a similar ability to replicate in the E1A positive HEK 293 cells, which are permissive for E3 /E3 deficient vector replication. The viruses spread through the cell cultures, causing extensive cytopathic effects (CPE) within three to seven days following infection, and produced progeny that were able to infect naive HEK 293 cells upon subsequent rounds of infection. The main difference noted between the vectors was that viruses encoding the cytokine gene constructs appeared to express the gfp reporter at levels lower than the Ad.gfp control virus. This difference in the levels of gfp expression is most likely a consequence of the configuration of the gfp gene as the second element of a bicistronic gene construct in the cytokine vectors, as opposed to the Ad.gfp vector, where the gfp gene is monocistronic. However, this did not appear to have any effect on viral propagation, as all viruses produced high titers following infection of HEK 293 cells.

The three recombinant viruses successfully transduced the HepG2 cells, as manifested by expression of the gfp reporter gene; however, a significant reduction in the levels of the reporter gene expression as compared to infection of the HEK 293 cells was observed (see FIG. 2, Primary (1°) “Infection Cycle”). This was especially notable for both the Ad.IL6gfp virus in HepG2 cells, where the gfp fluorescence levels were very weak. Obvious CPE resulting from the initial viral infection of HepG2 cells was evident 3-7 days post infection only in the cells infected with Ad.HIL6gfp (FIG. 2). Cultures infected with either Ad.gfp or Ad.IL6gfp appeared normal.

In order to determine whether the CPE observed in the HepG2 cell cultures was associated with active replication of the recombinant viruses, we attempted to retrieve newly formed viral particles from the infected cells shown in FIG. 2 (Primary (1°) “Infection Cycle”) by lysing the cells three days post infection. When extracts from the primary infected HepG2 cells were applied to fresh cultures of either the same cell type or HEK 293 cells, only extracts from cells infected by Ad.HIL6gfp proved capable of transferring the gfp activity (FIG. 2, Second (2°) “Infection Cycle”). Extracts from Ad.gfp or Ad.IL6gfp infected cells were not able to transfer the gfp activity resulting from the primary infection cycle, other than that resulting from infection of HEK 293 cells. Thus, the gfp activity observed in second infection cycle was most likely due to viral mediated de novo gfp expression, and not due to adsorption of gfp from the applied cell extract. Moreover, only extracts from Ad.HIL6gfp infected cells were able to produce notable and significant CPE upon subsequent rounds of infection in the E1A deficient cells. Cells incubated with extracts derived from the primary infection cycle by Ad.gfp or Ad.IL6gfp infected cells continued to grow well and maintained a normal morphology. Addition of purified recombinant HIL-6 protein to cell cultures at concentrations of up to 10 ng/ml, the highest concentration tested, did not produce any apparent toxic effect on the cells. Thus, the concurrent transfer of both gfp activity and CPE by extracts of Ad.HIL6gfp infected cells indicate that viral replication indeed occurred in the HepG2 cells.

### Example 2

### Cytopathic Effects of Ad.HIL6gfp Infection in HepG2 Cells

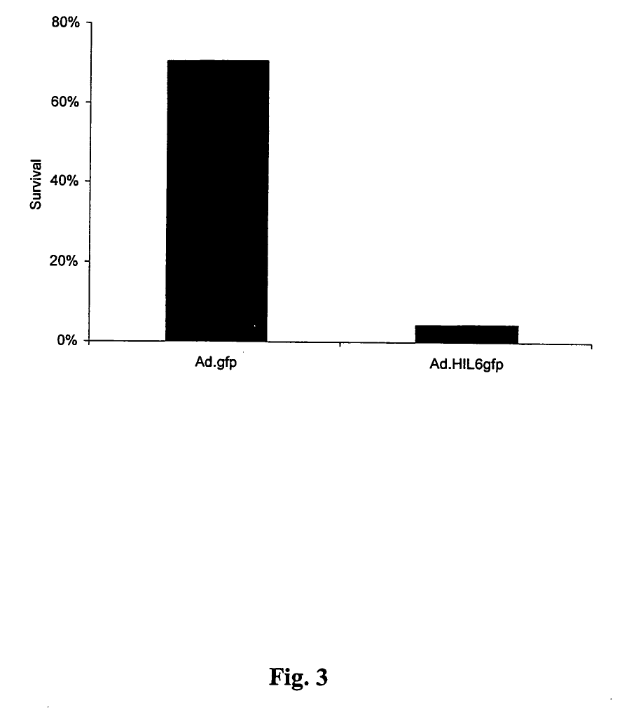

In order to quantify the cytopathic effect of the viral infection, HepG2 cells were infected with either Ad.HIL6gfp or Ad.gfp at an m.o.i of˜1, and were incubated with the viruses for two days and then assayed for surviving cells by methylene blue staining. The results of this analysis revealed that while approximately 80% of the Ad.gfp infected cells survived, only about 20% of the Ad.HIL6gfp infected cells were viable following the incubation period (FIG. 3).

### Example 3

### HIL-6 Protein Enables Replication of E1A Deficient Ad5 Vector in HepG2 Cells

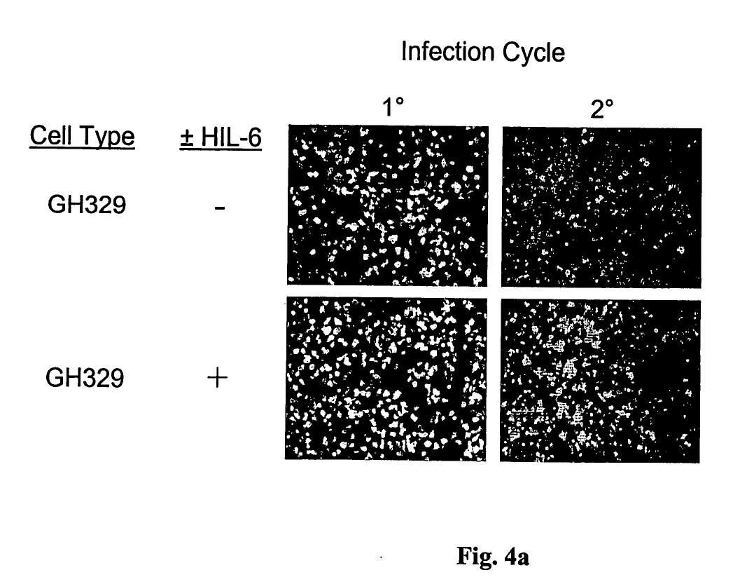

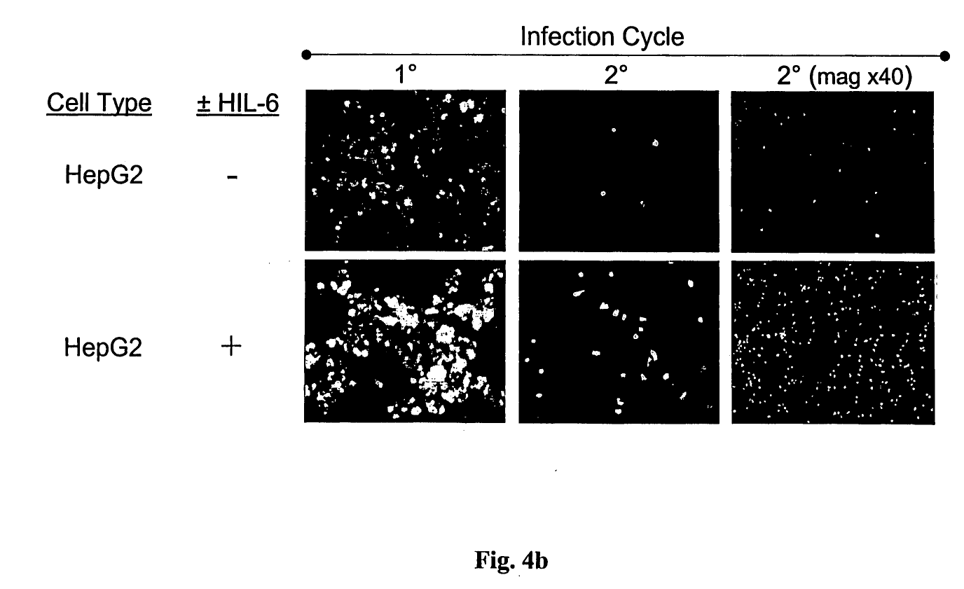

To confirm that HIL-6 enabled replication of an E1A deficient vector, HepG2 cells were infected with Ad.gfp, in the presence or absence of purified recombinant HIL-6 protein added to the culture media (FIG. 4). As shown in FIG. 4b, although far less robust than in the matching GH329 cell cultures (FIG. 4a), Ad.gfp infected HepG2 cells cultured for three days in the presence of exogenous HIL-6 protein displayed more gfp activity in comparison to identical cultures lacking HIL-6, suggesting that HIL-6 enabled viral replication. The HIL-6 protein supplement had no obvious effect on gfp expression or viral propagation in cultures of GH329 cells infected with Ad.gfp (FIG. 4). To verify that viral replication had indeed occurred in the HIL-6 supplemented HepG2 cell cultures, lysates from the primary infected cells were prepared and applied to HEK 293 cells. As anticipated, cells infected with Ad.gfp in the presence of HIL-6 produced significantly more viral progeny (˜15 fold more) compared to control Ad.gfp infected cells.

As disclosed in the specification of the present invention, a number of salient observations indicate that viral mediated HIL-6 expression enabled viral propagation in these cell lines. First, the concomitant transfer of gfp activity and CPE was unique to the Ad.HIL6gfp infected cultures and not apparent in cells, other than HEK 293 and GH329 cells, infected with either Ad.IL6gfp or the Ad.gfp. Secondly, because addition of purified recombinant HIL-6 protein was not toxic to the cells, the observed cytopathic effect of Ad.HIL6gfp can be attributed mainly to the effect of viral infection. Lastly, addition of exogenous HIL-6 protein to cultures of HepG2 cells enhanced the replication of the Ad.gfp control virus. Thus, HIL-6 expression is sufficient in order to compensate for the E1A deficiency in the viral vectors and allow viral propagation in these otherwise non-permissive cells.

Based on the previous observation that IL-6 induces a cellular function in HepG2 cells that functionally substitutes for the adenoviral E1A proteins, it was anticipated that HepG2 cells would also be permissive for replication of the Ad.IL6gfp vector. However, no evidence of Ad.IL6gfp replication in HepG2 cells was observed under the conditions used in this study. Two possible explanations may account for this observation. The effect of IL-6 on HepG2 cells observed in previous studies rests, in part, upon the fact that HepG2 cells express significant levels of sIL-6R, which is available to complex with exogenously added IL-6. In the present study, the Ad.IL6gfp virus was applied to the HepG2 cell cultures at low titers (m.o.i. 1). As such the viral transgene expression and the resulting IL-6 levels may have been insufficient to induce the E1A-substituting activity to levels where effective viral replication is made possible. Alternatively, the HepG2 cell line used may, through spontaneous changes in gene expression patterns, produce insufficient levels of IL-6R to support the IL-6 induced E1A complementing activity. However, in either case, Ad.HIL6gfp mediated HIL-6 expression remedied all deficiencies, and successfully supported vector replication.

These results suggest that a self-contained IL-6 autocrine arc carried by E1A deficient adenoviral vectors, such as Ad.HIL6gfp, will enable viral propagation and produce oncolytic activity in a wide variety of tumor cells. As such, viral vectors encoding designer cytokines such as HIL-6 may have a broad utility as therapeutic agents for treating cancers of various types, either as a single agent, or applied in combination with other therapeutic strategies.

### Example 4

### Anti-Tumor Effects of Ad.HIL6gp In Vivo

The enhanced cytopathic effect of Ad.HIL6gfp observed in HepG2 cells in vitro, has suggested that in vivo tumor growth may also be diminished by Ad.HIL6gfp infection. In order to examine this hypothesis, a subcutaneous xenograft tumor model involving injection of HepG21uc cells in BALB/C nude mice is developed. HepG21uc cells are derived from the parental HepG2 cells by genetic manipulation to generate constitutive expression of firefly luciferase in the cells, thus causing them to emit light in the presence of the luciferase substrate, luciferin (17). HepG21uc tumors are treated by intra-tumoral injection of Ad.HIL6gfp, AdIL6gfp, or Ad.gfp in doses of 10⁷-10⁹ TU (transducing units). Tumor growth as indicated by quantitative in situ light emission in the presence of luciferin measured using a cooled charged coupled device (CCCD) camera, and by caliper measurement of tumor volume is performed to monitor the oncolytic effect of the recombinant virus administration. Animals are also monitored for survival.

### Example 5

### Analysis of an Ad.HIL6gfp Induced Immune mediated Anti-Tumor Response

IL-6/sIL-6R has been shown to mediate leukocyte recruitment, in part via the expression of certain chemokines (IL-8, MCP-1 and MCP-3) and adhesion molecules (ICAM-1 and VCAM-1). Thus, in the context of an immune competent host, the Ad.HIL6gfp induced anti-neoplastic activity may involve both a viral mediated oncolytic component and an immune mediated component. We have, therefore, hypothesized that HIL-6 expression in Ad.HIL6 treated tumors would lead to substantially higher levels of leukocyte infiltration to the tumor and, possibly, enhanced immune mediated anti-tumor activity. In order to test the effect of HIL-6 mediated immune cell infiltration, subcutaneous tumors based on murine liver derived BNL1ME.A7luc tumor cells (cells that constitutively express firefly luciferase, thus emit light in the presence of the luciferase substrate, luciferin) implanted in syngeneic immune competent BALB/C mice or BALB/C nude mice are treated by injection of either Ad.HIL6gfp (10⁸ TU) or control viruses. The effect of viral infection on tumor growth kinetics is monitored by in situ luciferase expression and by growth in tumor volume. To analyze the effect of Adeno mediated HIL-6 expression on immune cell trafficking into tumor nodules, infiltrating immune cells are extracted from excised tumor nodules and characterized by common immune associated cell surface markers (CD3, CD4/CD8, CD56, CD14) by FACS analysis.

To analyze for the presence of tumor specific immune activity in the isolated tumor associated immune cells, ⁵¹Cr loaded BNL1ME.A7luc tumor cells are co-cultured for 24 hours with increasing numbers of the isolated immune cells and followed by analysis of ⁵¹Cr release to the culture medium.

To establish whether Ad.HEL6gfp treatment can induce a long-term anti-tumor immune response, an adoptive immune transfer experiment is performed. Total spleen cells from syngeneic BALB/C mice carrying an implanted BNL1ME.A7luc tumor burden and treated with either Ad.HIL6 or control vector are isolated and transferred to naive animals prior to tumor cell implantation. Control animals are treated with total spleen cells isolated from mice treated with either Ad.HIL6 or control vector or from untreated mice. The animals are then scored for the ability to reject implanted BNL1ME.A7luc tumor cells and for survival.

It will be appreciated by persons skilled in the art that the present invention is not limited by what has been particularly shown and described herein above. Rather the scope of the invention is defined by the claims, which follow.

References Cited:

1. Bischoff, J. R., Kim, D. H., Williams, A., Heise, C., Horn, S., Muna, M., Ng, L., Nye, J. A., Sampson-Johannes, A., Fattaey, A., and McCormick, F. An adenovirus mutant that replicates selectively in p53-deficient human tumor cells, Science. 274: 373-6, 1996.
2. Russell, W. C. Update on adenovirus and its vectors, J Gen Virol. 81 Pt 11: 2573-604, 2000.
3. Spergel, J. M. and Chen-Kiang, S. Interleukin 6 enhances a cellular activity that functionally substitutes for E1A protein in transactivation, Proc Natl Acad Sci U S A. 88: 6472-6, 1991.
4. Spergel, J. M., Hsu, W., Akira, S., Thimmappaya, B., Kishimoto, T., and Chen-Kiang, S. NF-IL6, a member of the C/EBP family, regulates E1A-responsive promoters in the absence of E1A, J Virol. 66: 1021-30., 1992.
5. Rancourt, C., Piche, A., Gomez-Navarro, J., Wang, M., Alvarez, R. D., Siegal, G. P., Fuller, G. M., Jones, S. A., and Curiel, D. T. Interleukin-6 modulated conditionally replicative adenovirus as an antitumor/cytotoxic agent for cancer therapy, Clin Cancer Res. 5: 43-50, 1999.
6. Peters, M., Muller, A. M., and Rose-John, S. Interleukin-6 and soluble interleukin-6 receptor: direct stimulation of gp130 and hematopoiesis, Blood. 92: 3495-504, 1998.
7. Fischer, M., Goldschmitt, J., Peschel, C., Brakenhoff, J. P., Kallen, K. J., Wollmer, A., Grotzinger, J., and Rose-John, S. I. A bioactive designer cytokine for human hematopoietic progenitor cell expansion, Nat Biotechnol. 15: 142-5. 1997.
8. Peters, M., Blinn, G., Solem, F., Fischer, M., Meyer zum Buschenfelde, K. H., and Rose-John, S. In vivo and in vitro activities of the gp130 -stimulating designer cytokine Hyper-IL-6, J Immunol. 161: 3575-81, 1998.
9. Hecht, N., Pappo, O., Shouval, D., Rose-John, S., Galun, E., and Axelrod, J. H. Hyper-IL-6 gene therapy reverses fulminant hepatic failure, Mol Ther. 3: 683-7, 2001.
10. Tanaka, F., Abe, M., Akiyoshi, T., Nomura, T., Sugimachi, K., Kishimoto, T., Suzuki, T., Okada, M. The anti-human tumor effect and generation of human cytotoxic T cells in SCID mice given human peripheral blood lymphocytes by the in vivo transfer of the interleukin-6 gene using adenovirus vector. Cancer Res. 57:1335-43, 1997.
11. Felzmann, T., Ramsy, W. J., Blaese, R. M. Characterization of the antitumor immune response generated by treatment of murine tumors with recombinant adenoviruses expressing HSVtk, IL-2, IL-6 or B7-1. Gene Ther. 4:1322-9, 1997.
12. Graham, F. L., Smiley, J., Russell, W. C., and Nairn, R. Characteristics of a human cell line transformed by DNA from human adenovirus type 5, J Gen Virol. 36: 59-74., 1977.
13. Gao, G. P. Engdahl, R. K. and Wilson, J. M. A cell line for high-yield production of E3 -deleted adenovirus vectors without the emergence of replication-competent virus, Hum Gene Ther 11: 213-219. 2000
14. Knowles, B. B., Howe, C. C., and Aden, D. P. Human hepatocellular carcinoma cell lines secrete the major plasma proteins and hepatitis B surface antigen, Science. 209: 497-9. 1980.
15. Nakabayashi, H., Taketa, K., Miyano, K., Yamane, T., and Sato, J. Growth of human hepatoma cells lines with differentiated functions in chemically defined medium, Cancer Res. 42: 3858-63. 1982.
16. He, T. C., Zhou, S., da Costa, L. T., Yu, J., Kinzler, K. W., and Vogelstein, B. A simplified system for generating recombinant adenoviruses, Proc Natl Acad Sci USA. 95: 2509-14, 1998.
17. Honigman, A., Zeira, E., Ohana, P., Abramovitz, R., Tavor, E., Bar, I., Zilberman, Y., Rabinovsky, R., Gazit, D., Joseph, A., Panet, A., Shai, E., Palmon, A., Laster, M., and Galun, E. Imaging transgene expression in live animals. Mol Ther, 4: 239-249. 2001.

1. A method for treating a neoplastic disease in a subject comprising administering to the subject a therapeutically effective amount of a recombinant replication defective adenovirus vector exerting oncolytic activity, the vector comprising a DNA sequence encoding an IL-6/soluble IL-6 receptor (sIL-6R) complex or a biologically active variant thereof, the DNA sequence being operably linked to a promoter.

2. The method according to claim 1, wherein the recombinant adenovirus vector is selected from the group consisting of partially or fully deleted E3 adenovirus vectors, partially or fully deleted E3 adenovirus vectors, partially or fully deleted E4 adenoviral vectors, and combinations thereof.

3. The method according to claim 1, wherein the adenovirus vector is selected from the group consisting of Ad2 and Ad5 derived vectors.

4. The method according to claim 1, wherein the IL-6/sIL-6R complex is hyper-IL-6 (HIL-6).

5. The method according to claim 4, wherein the recombinant adenovirus vector encoding HIL-6 has partially or fully deleted E1 gene.

6. The method according to claim 4, wherein the recombinant adenovirus vector encoding HIL-6 has partially or fully deleted E3 gene.

7. The method according to claim 4, wherein the recombinant adenovirus vector encoding HIL-6 has partially or fully deleted E1 and E3 genes.

8. The method according to claim 1, wherein the recombinant adenovirus vector further comprising a DNA sequence encoding at least one other non-viral protein.

9. The method according to claim 8, wherein the non-viral protein simulates an extracellular or intracellular effect of IL-6/sIL-6R complex by enhancing gp130 signaling, or any of the down-stream molecular events resulting from gp130 signal transduction, or a variant thereof.

10. The method according to claim 9, wherein the non-viral protein is selected from the group consisting of STAT-3 and NF-IL6.

11. The method according to claim 8, wherein the non-viral protein is selected from the group consisting of cytokines, suicide factors, transcription factors, and biologically active variants thereof.

12. The method according to claim 11, wherein the cytokine is a cytokine having a receptor complex comprising gp130.

13. The method according to claim 1, wherein the recombinant adenovirus vector is altered to enter through a cell receptor other than those native to adenoviruses.

14. The method according to claim 1, further comprising treatment with an additional anti-neoplastic therapeutic agent selected from an immunosuppressive agent, a chemotherapeutic agent and an anti-proliferative agent.

15. The method according to claim 14, wherein the vector and agent are administered substantially at the same time in a single composition.

16. The method according to claim 14, wherein the vector and agent are administered sequentially in separate compositions.

17. The method according to claim 1, wherein the administering is intravenously, intraarterially, intraperitoneally, subcutaneously, intradermally, intramuscularly injecting of the adenoviral vector, injecting directly into a tumor mass, or injecting locally in the proximity of a tumor mass.

18. The method according to claim 17, wherein the administering is direct injecting of the adenoviral vector into the tumor mass.

19. The method according to claim 1, wherein the neoplastic disease is selected from benign solid tumors, malignant solid tumors, benign proliferative diseases of the blood, and malignant proliferative diseases of the blood.

20. The method according to claim 19, wherein the neoplastic disease is liver cancer.

21. A method for treating a neoplastic disease in a subject comprising administering a therapeutically effective amount of a first recombinant replication defective adenovirus vector comprising a DNA sequence encoding IL-6 or a biologically active variant thereof operably linked to a promoter; and administering a second recombinant replication defective adenovirus vector, the second vector comprising a DNA sequence encoding sIL-6R or a biologically active variant thereof operably linked to a promoter, and wherein the first and second vectors exert oncolytic activity.

22. The method according to claim 21, further comprising treatment with an additional anti-neoplastic therapeutic agent selected from the group consisting of an immunosuppressive agent, a chemotherapeutic agent and an anti-proliferative agent.

23. A method for treating a neoplastic disease in a subject comprising administering to the subject a therapeutically effective amount of transfected eukaryotic cells, said transfected cells being transduced with a recombinant replication defective adenovirus vector exerting oncolytic activity, said vector comprising a DNA sequence encoding an IL-6/sIL-6R complex or a biologically active variant thereof, said DNA sequence being operably linked to a promoter.

24. The method according to claim 23, wherein the administering is intravenously, intraarterially, intraperitoneally, subcutaneously, intradermally, intramuscularly injecting of the adenoviral vector, injecting directly into a tumor mass, or injecting locally in the proximity of a tumor mass.

25. The method according to claim 24, wherein the administering is direct injecting of the adenoviral vector into the tumor mass.

26. The method according to claim 23, wherein the neoplastic disease is selected from benign solid tumors, malignant solid tumors, benign proliferative diseases of the blood, and malignant proliferative diseases of the blood.

27. The method according to claim 26, wherein the neoplastic disease is liver cancer.

28. A method for treating a neoplastic disease in a subject comprising administering to the subject a therapeutically effective amount of a recombinant viral vector exerting oncolytic activity, the vector comprising a DNA sequence encoding an IL-6/sIL-6R complex or a biologically active variant thereof is selected from adeno associated virus, Herpes virus, Measles virus, and Newcastle Disease virus.

29. A recombinant replication defective adenovirus vector exerting oncolytic activity, said vector selected from the group consisting of partially or fully deleted E3 adenovirus vectors, partially or fully deleted E3 adenovirus vectors, partially or fully deleted E4 adenovirus vectors, or combinations thereof, said vector further comprising a DNA sequence encoding an IL-6/soluble IL-6receptor (sIL-6R) complex or a biologically active variant thereof operably linked to a promoter.

30. The recombinant replication defective adenovirus vector according to claim 29, wherein the adenovirus vector is selected from the group consisting of Ad2 and Ad5 derived vectors.

31. The recombinant replication defective adenovirus vector according to claim 30, wherein the IL-6/sIL-6R complex is hyper IL-6 (HIL-6).

32. The recombinant replication defective adenovirus vector according to claim 31 is Ad.HIL6 or Ad.HIL6gfp.

33. The recombinant replication defective adenovirus vector according to claim 29, further comprising a DNA sequence encoding at least one other non-viral protein.

34. A pharmaceutical composition for treating a neoplastic disease comprising as an active ingredient a recombinant replication defective adenovirus vector exerting oncolytic activity, the vector selected from the group consisting of partially or fully deleted E1 adenovirus vectors, partially or fully deleted E3 adenovirus vectors, partially or fully deleted E4 adenovirus vectors, or combinations thereof, said vector further comprising a DNA sequence encoding an IL-6/soluble IL-6receptor (SIL-6R) complex or a biologically active variant thereof operably linked to a promoter.

35. The composition according to claim 34, wherein the adenovirus vector is selected from the group consisting of Ad2 and Ad5 derived vectors.

36. The composition according to claim 34, wherein the IL-6/sIL-6R complex is hyper IL-6(HIL-6).

37. The composition according to claim 34, wherein the recombinant replication defective adenovirus vector is Ad. HIL6 or Ad. HIL6gfp.

38. The composition according to claim 34, wherein the recombinant adenovirus vector further comprising a DNA sequence encoding at least one other non-viral protein.

39. The composition according to claim 34, further comprising a pharmaceutical acceptable carrier.
